(19) United States
(12) Patent Application Publication
(10) Pub. No.: US 2021/0196114 A1
(43) Pub. Date: Jul. 1, 2021

(54) OPHTHALMIC DEVICE

(71) Applicant: NIKON CORPORATION, Tokyo (JP)

(72) Inventors: Shota MIYAZAKI, Fujisawa-shi (JP); Ken TOMIOKA, Yokohama-shi (JP)

(73) Assignee: NIKON CORPORATION, Tokyo (JP)

(21) Appl. No.: 17/130,427

(22) Filed: Dec. 22, 2020

(30) Foreign Application Priority Data
Dec. 27, 2019 (JP) 2019-237796

(51) Int. Cl. A61B 3/024; A61B 3/10; A61B 3/00

(52) U.S. Cl. CPC A61B 3/024; A61B 3/102; A61B 3/0091; A61B 3/0016; A61B 3/0075

(57) ABSTRACT

An ophthalmic device including a vision field testing section including a target presentation section and a vision field test optical system and configured to perform a vision field test on an examination eye, a measuring section including a measurement optical system to perform optical coherence tomography measurements on the examination eye, and a measurement unit configured to generate a tomographic image of the examination eye, an optical path combining section provided between the examination eye and the target presentation section and configured to combine a first optical path of the vision field test optical system and a second optical path of the measurement optical system, a first lens provided between the optical path combining section and the target presentation section and configured to perform diopter adjustment, a second lens provided between the optical path combining section and a position of the examination eye or the measurement unit, and employed in focus adjustment, a first driving section configured to move the first lens, a second driving section configured to move the second lens, and a control section configured to control the first driving section and the second driving section.

### CROSS-REFERENCE TO RELATED APPLICATIONS

This application claims priority from Japanese Patent Application No. 2019-237796, filed Dec. 27, 2019, the disclosure of which is incorporated herein by reference in its entirety.

### BACKGROUND

### Technical Field

The technology disclosed herein relates to an ophthalmic device.

### Related Art

Perimeters such as that described in U.S. Pat. No. 9,155,464 that performs vision field based on an examinee's response to seeing presented targets, are known.

### SUMMARY

An ophthalmic device of a first aspect of the technology disclosed herein comprising: a vision field testing section including a target presentation section and a vision field test optical system and configured to perform a vision field test on an examination eye; a measuring section including a measurement optical system to perform optical coherence tomography measurements on the examination eye, and a measurement unit configured to generate a tomographic image of the examination eye; an optical path combining section provided between the examination eye and the target presentation section and configured to combine a first optical path of the vision field test optical system and a second optical path of the measurement optical system; a first lens provided between the optical path combining section and the target presentation section and configured to perform diopter adjustment; a second lens provided between the optical path combining section and either a position of the examination eye or the measurement unit, and employed in focus adjustment; a first driving section configured to move the first lens; a second driving section configured to move the second lens; and a control section configured to control the first driving section and the second driving section.

### BRIEF DESCRIPTION OF THE DRAWINGS

FIG. 1 is a block diagram of an ophthalmic system 100 of a first exemplary embodiment.

FIG. 2 is a schematic configuration diagram illustrating an overall configuration of an ophthalmic device 110A of the first exemplary embodiment.

FIG. 3 is a block diagram of a configuration of an electrical system of the ophthalmic device 110A of the first exemplary embodiment.

FIG. 4 is a block diagram of a configuration of an electrical system of a server 140 of the first exemplary embodiment.

FIG. 5 is a functional block diagram of a CPU 22 of the ophthalmic device 110A of the first exemplary embodiment.

FIG. 6 is a flowchart of a vision field test program and an OCT image acquisition processing program of the first exemplary embodiment.

FIG. 7 is a flowchart of processing to set measurement conditions at step 304 of FIG. 6.

FIG. 8 is a diagram illustrating a measurement condition input screen 400.

FIG. 9 is a diagram illustrating presentation of a fixation target.

FIG. 10 is a diagram illustrating presentation of a target 502 at a position n on a hemispherical inner face of a target presentation section 52.

FIG. 11 is a concept diagram of OCT image data 502 IOCTD of a retina obtained based on position 5021 on a retina 510.

FIG. 12 is a diagram illustrating a visual field defect map 510M.

FIG. 13 is a diagram illustrating a report display screen 600.

FIG. 14 is a schematic configuration diagram illustrating an overall configuration of an ophthalmic device 110B of a second exemplary embodiment.

FIG. 15 is a schematic configuration diagram illustrating an overall configuration of an ophthalmic device 110C of a third exemplary embodiment.

FIG. 16 is a schematic configuration diagram illustrating an overall configuration of an ophthalmic device 110D of a fourth exemplary embodiment.

FIG. 17 is a flowchart of a vision field test program and an OCT image acquisition processing program of a first modified example.

FIG. 18 is a diagram illustrating a fixation target when positioned on a horizontal plane.

FIG. 19 is a diagram illustrating a fixation target when positioned above a horizontal plane.

FIG. 20 is a diagram illustrating a fixation target when positioned below a horizontal plane.

### DETAILED DESCRIPTION

Detailed description follows regarding exemplary embodiments of the present invention, with reference to the drawings.

### First Exemplary Embodiment

Explanation follows regarding an ophthalmic system including an ophthalmic device according to a first exemplary embodiment of the present invention, with reference to the drawings.

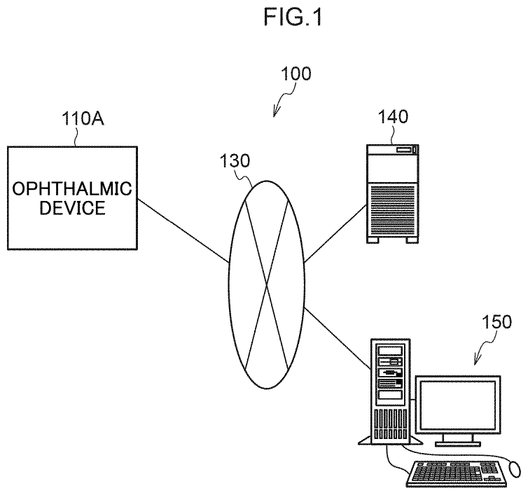

Description follows regarding a configuration of an ophthalmic system 100, with reference to FIG. 1. As illustrated in FIG. 1, the ophthalmic system 100 includes an ophthalmic device 110A, a management server device (referred to hereafter as “server”) 140, and an image display device (referred to hereafter as “viewer”) 150. The ophthalmic device 110A acquires vision field measurements and tomographic images. The server 140 stores plural vision field test results obtained by the ophthalmic device 110A and tomographic images obtained by fundus imaging, and stores these in association with IDs of examinees (also referred to as patients). The viewer 150 displays images and the like acquired from the server 140.

The ophthalmic device 110A, the server 140, and the viewer 150 are coupled together over a network 130.

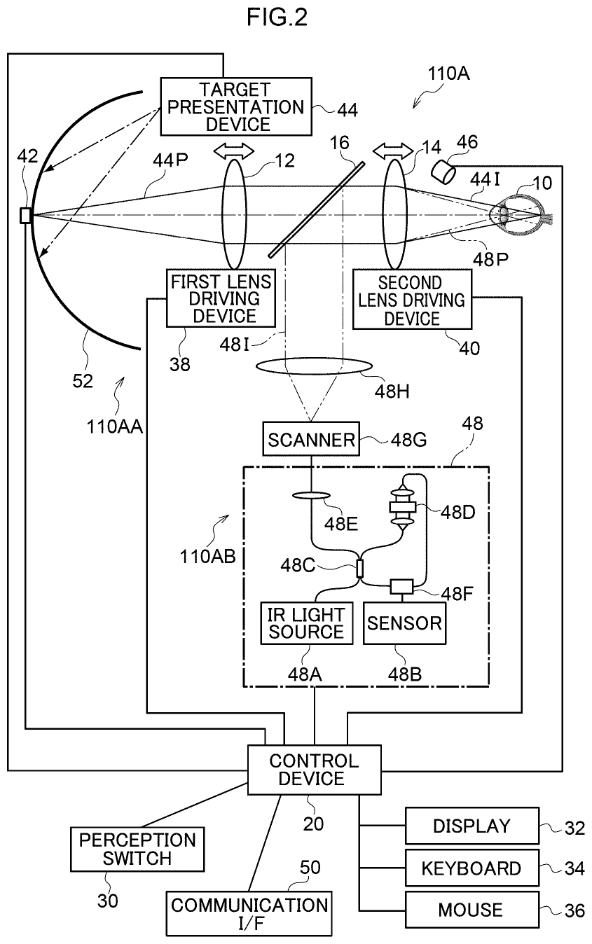

Next, explanation follows regarding a configuration of the ophthalmic device 110A, with reference to FIG. 2.

Note that in cases in which the ophthalmic device 110A is installed on a horizontal plane, the horizontal direction is denoted an X direction, a direction perpendicular to the horizontal plane is denoted a Y direction, and a direction connecting the center of the pupil at an anterior eye portion and the center of the eyeball of an examination eye 10 is denoted a Z direction. The X direction, the Y direction, and the Z direction are thus mutually perpendicular directions.

FIG. 2 illustrates the ophthalmic device 110A. The ophthalmic device 110A includes a vision field testing section 110AA, a first lens 12, an optical path combining section 16, a second lens 14, an external camera 46, and a measuring section 110AB. The ophthalmic device 110A also includes a perception switch 30, and, serving as input/output devices, a display 32, a keyboard 34, a mouse 36, and a communication IF 50.

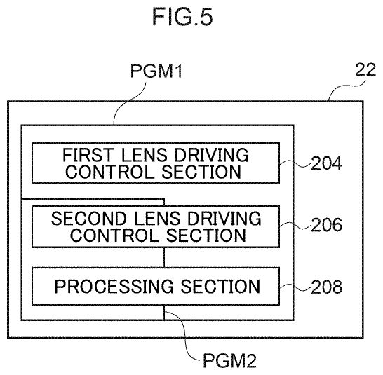

The vision field testing section 110AA performs a vision field test on the examination eye 10. The vision field testing section 110AA is a section for performing vision field on the examination eye 10, and includes a target presentation section 52, a vision field testing optical system (52, 12, 16, 14, 38, 40), a target presentation device 44, and the perception switch 30. The target presentation section 52 is formed by a dome having a hemispherical inner face as a reflection surface. The target presentation device 44 presents targets (specifically, projects light) at plural different positions on the hemispherical inner face of the target presentation section 52 at different timings according to a vision field program, described later. The targets are projected onto the dome by the vision field testing optical system (52, 12, 16, 14, 38, 40). The light of the presented targets reaches the retina of the examination eye 10 via the target presentation section 52, the first lens 12, the optical path combining section 16, and the second lens 14. An optical path 44P of the light of the targets is formed by the target presentation section 52, the first lens 12, the optical path combining section 16, and the second lens 14. The first lens 12 is driven by a first lens driving device 38 and the second lens 14 is driven by a second lens driving device 40, based on examination eye refractive index information, focus control information, and the like. The first lens driving device 38 and the second lens driving device 40 are driven under control of a control device 20. The control device 20 operates under control of a processing section 208 (see FIG. 5) based on the vision field program, and generates a vision field map based on the target projection positions and detection signals output from the perception switch 30.

The measuring section 110AB includes a measurement optical system (48H, 14) to perform optical coherence tomography (OCT) measurements of the examination eye 10, a scanner 48G, and an OCT imaging device 48 to generate tomographic images (OCT images) of the examination eye 10.

The measuring section 110AB is an example of a “measurement section” of technology disclosed herein.

The OCT imaging device 48 includes a light source 48A, a sensor (detection element) 48B, a first optical coupler 48C, a reference optical system 48D, a collimator lens 48E, and a second optical coupler 48F.

The light emitted from the light source 48A is split by the first optical coupler 48C. Part of the split light serves as measurement light, and is converted into a parallel light beam by the collimator lens 48E before being incident to the scanner 48G. The measurement light is scanned by the scanner 48G in both the X direction and the Y direction. The scanned light is irradiated onto the fundus of the examination eye 10 through an optical system 48H, the optical path combining section 16, the second lens 14, and a pupil. The measurement light that is reflected by the fundus is incident to the OCT imaging device 48 through the second lens 14, the optical path combining section 16, the optical system 48H, and the scanner 48G, and is incident to the second optical coupler 48F through the collimator lens 48E and the first optical coupler 48C. The scanner 48G operates under control of the control device 20 based on an OCT acquisition range. Moreover, the second lens 14 is driven by the second lens driving device 40 using focus control information and the like.

Note that the optical system 48H performs optical path length adjustment so as to form light into an image at the center of the lens of the examination eye 10 when the light is being scanned by the scanner 48G.

The other part of the light emitted from the light source 48A and split by the first optical coupler 48C serves as reference light, and is incident to the reference optical system 48D, and incident to the second optical coupler 48F through the reference optical system 48D.

This light that is incident to the second optical coupler 48F, namely the measurement light reflected by the fundus and the reference light, is combined by the second optical coupler 48F to generate interference light. The interference light is picked up by the sensor 48B. The control device 20 operates under control of the processing section 208 (see FIG. 5) based on an OCT image acquisition processing program so as to generate OCT data and generate OCT images, such as tomographic images, en-face images, and the like based on detection signals detected by the sensor 48B.

Note that although in the present exemplary embodiment an example is given in which the light source 48A is a swept-source OCT (SS-OCT) type, the light source 48A employed may be based on various OCT systems, such as from a spectral-domain OCT (SD-OCT) or from a time-domain OCT (TD-OCT) system.

Note that the OCT imaging device 48 is not necessarily installed at a right angle to the eyeball optical axis of the examination eye, and the use of mirrors, optical fibers, or the like allow installation at various positions.

The light (measurement light) from the OCT imaging device 48 reaches the retina of the examination eye 10 via the optical path combining section 16 and the second lens 14, as described above. Light from the OCT imaging device 48 is formed into an optical path 48P by the OCT imaging device 48, the optical path combining section 16, and the second lens 14. The optical path combining section 16 is provided between the examination eye 10 and the target presentation section 52, and combines the optical path 44P of the vision field testing optical system and the optical path 48P of the measurement optical system of the OCT imaging device 48. The optical path combining section 16 is configured by a flat plane half-mirror.

The first lens 12 is provided on the optical path 44P between the optical path combining section 16 and the target presentation section 52, and performs diopter adjustment (vision correction) based on an eyesight correction value for the examination eye 10. The diopter adjustment is executed by driving the first lens driving device 38 according to an input eyesight correction value. The first lens 12 may be configured by a variable refractive index member such as a liquid lens, and in such cases adjustments may be accommodated by changing the refractive index according to the eyesight correction value, without changing the position of the first lens 12. Moreover, a configuration may be adopted in which both the position and the refractive index of a liquid lens are changed.

The optical path combining section 16 is not limited to being a flat plane half-mirror, and light may be introduced to the examination eye 10 using a non-flat plane (spherical surface) half-mirror having a spherical surface, an aspherical surface, or the like. In cases in which the surface of the optical path combining section 16 is not a flat plane, the position of the first lens 12 is adjusted to counter the spherical surface of the non-flat plane optical path combining section 16.

The second lens 14 is provided between the optical path combining section 16 and the position of the examination eye 10, and performs adjustment to focus the measurement light on the fundus in order to acquire an OCT image. Focus adjustment is likewise performed during vision field testing.

The position of the first lens 12 is adjusted after adjusting the position of the second lens 14.

The ophthalmic device 110A includes the first lens driving device 38 to move the first lens 12, the second lens driving device 40 to move the second lens 14, and the control device 20 to control both the first lens driving device 38 and the second lens driving device 40.

The first lens driving device 38 is an example of a “first driving section” of the technology disclosed herein. The second lens driving device 40 is an example of a “second driving section” of the technology disclosed herein. The control device 20 is an example of a “control section” of technology disclosed herein.

The control device 20 drives the first lens driving device 38 and the second lens driving device 40 so as to adjust the positions of the first lens 12 and the second lens 14 when vision field testing is being performed, as described later. The control device 20 moves at least the first lens 12 based on an eyesight correction value for the examination eye 10 when vision field testing is being performed. The control device 20 drives the second lens driving device 40 so as to adjust the position of the second lens 14 during tomographic image generation.

The ophthalmic device 110A includes a fixation target presentation section 42. The fixation target presentation section 42 presents a fixation target commonly employed during both vision field testing and tomographic image generation.

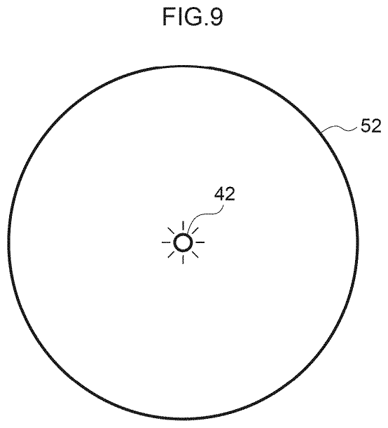

A hole is provided at a position at the center of the hemispherical inner face of the target presentation section 52 (see FIG. 9), namely, on the optical axis of the examination eye 10, and the fixation target presentation section 42 is provided at a position corresponding to this provided hole. Thus when the fixation target presentation section 42 is illuminated light of the fixation target travels from the fixation target presentation section 42, through the hole in the target presentation section 52, and through the first lens 12, the optical path combining section 16, and the second lens 14 before reaching the examination eye 10. The examinee fixates on the fixation target. The optical axis of the examination eye 10 is thereby aligned with the fixation target.

Between the optical path combining section 16 and the position of the examination eye 10, the optical path 44P and the optical path 48P share a common optical path. The second lens 14 is positioned on this common optical path.

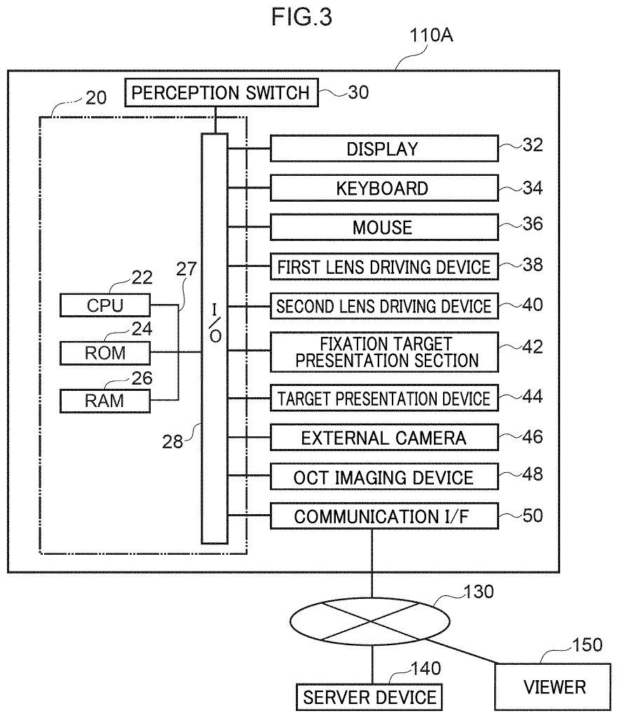

FIG. 3 illustrates an electrical system of the ophthalmic device 110A. As illustrated in FIG. 3, the ophthalmic device 110A includes the control device 20. The control device 20 is configured by a computer. The control device 20 includes a central processing unit (CPU) 22, read only memory (ROM) 24, random access memory (RAM) 26, and an input/output (I/O) port 28. These units from the CPU 22 to the input/output (I/O) port 28 are connected together through a bus 27. The perception switch 30, the display 32, the keyboard 34, the mouse 36, the first lens driving device 38, the second lens driving device 40, the fixation target presentation section 42, the target presentation device 44, the external camera 46, the OCT imaging device 48, and the communication interface (I/F) 50 are all connected to the input/output (I/O) port 28.

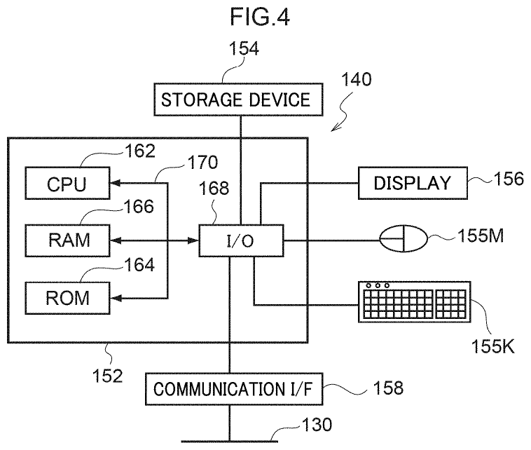

Next, explanation follows regarding a configuration of an electrical system of the server 140, with reference to FIG. 4. As illustrated in FIG. 4, the server 140 includes a computer main unit 152. The computer main unit 152 includes a CPU 162, RAM 166, ROM 164, and an input/output (I/O) port 168. A storage device 154, a display 156, a mouse 155M, a keyboard 155K, and a communication interface (I/F) 158 are connected to the input/output (I/O) port 168. The storage device 154 is configured, for example, by non-volatile memory. The input/output (I/O) port 168 is connected to the network 130 through the communication interface (I/F) 158. The server 140 is accordingly able to communicate with the ophthalmic device 110A and with the viewer 150.

The server 140 stores various data received from the ophthalmic device 110A in the storage device 154.

The configuration of the electrical system of the viewer 150 is similar to the configuration of the electrical system of the server 140, and so further explanation thereof will be omitted.

Next, explanation follows regarding various functions implemented by the CPU 22 of the ophthalmic device 110A executing vision field test and OCT image acquisition processing programs, with reference to FIG. 5. The vision field test and OCT image acquisition processing programs include a first lens driving control function, a second lens driving control function, and a processing function. More specifically, the vision field test and OCT image acquisition processing programs include a vision field test program PGM1 and an OCT image acquisition processing program PGM2. The vision field test program PGM1 includes a first lens driving control function, a second lens driving control function, and a processing function. The OCT image acquisition processing program PGM2 includes a second lens driving control function and a processing function. The CPU 22 functions as a first lens driving control section 204, a second lens driving control section 206, and a processing section 208, as illustrated in FIG. 5, by the CPU 22 executing the vision field test and OCT image acquisition processing programs (PGM1, PGM2) including these functions.

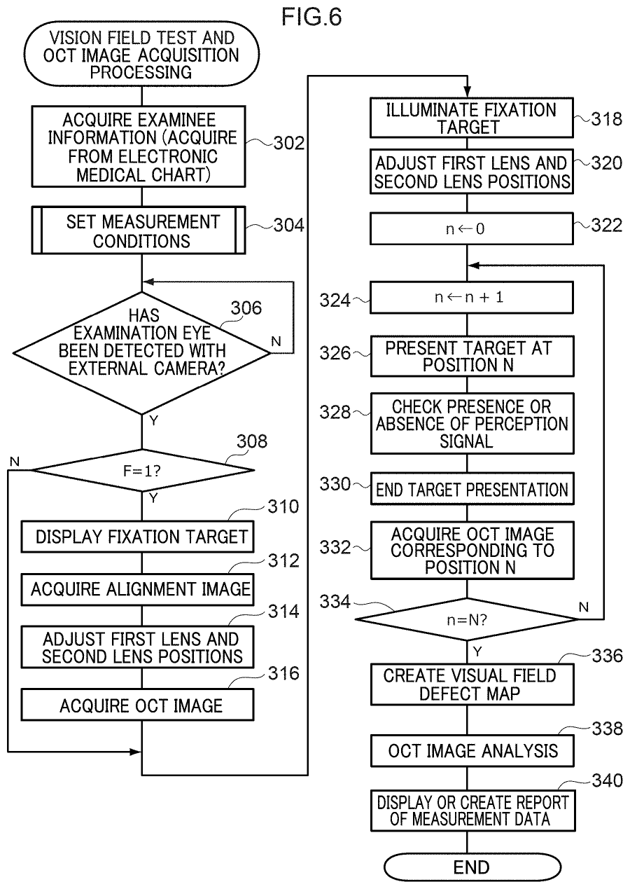

Detailed explanation next follows regarding vision field test and OCT image acquisition processing performed by the ophthalmic device 110A, with reference to FIG. 6. FIG. 6 illustrates a flowchart for the vision field test and OCT image acquisition processing programs. The CPU 22 of the ophthalmic device 110A implements the vision field test and OCT image acquisition processing illustrated in the flowchart of FIG. 6 by executing the vision field test and OCT image acquisition processing programs.

At step 302, based on a patient ID input by a user or operator, the processing section 208 acquires examinee information corresponding to the patient ID from the server 140 through the communication interface (I/F) 50. The examinee information includes examinee name, examinee ID, past data about the examinee (vision field test result data and OCT image data), and the like. Note that in cases in which an electronic medical chart device stored with examinee information is provided in addition to the server 140, the processing section 208 acquires the examinee information from this electronic medical chart device.

At step 304, the processing section 208 sets measurement conditions for performing vision field test with the vision field testing section 110AA, and for performing OCT imaging with the measuring section 110AB.

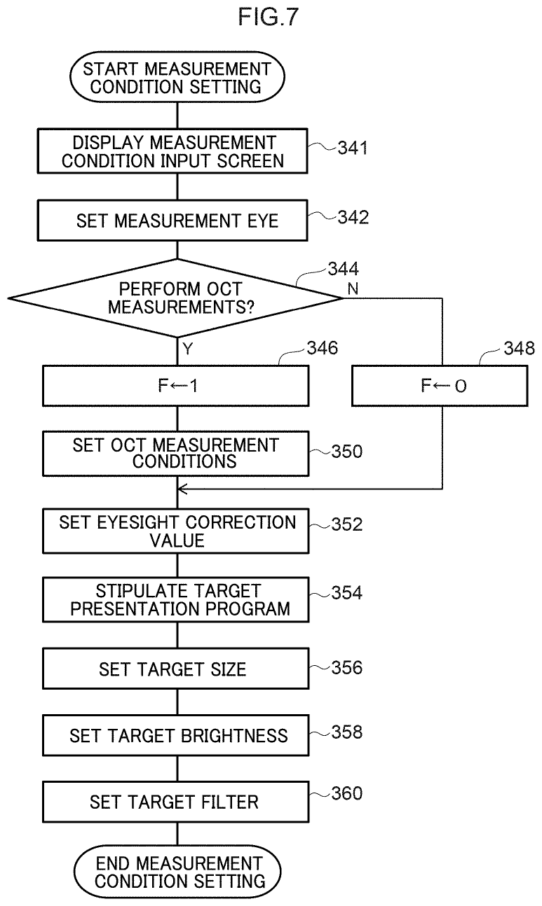

Explanation follows regarding processing to set the measurement conditions at step 304 of FIG. 6, with reference to FIG. 7. FIG. 7 illustrates a flowchart of processing to set the measurement conditions at step 304 of FIG. 6.

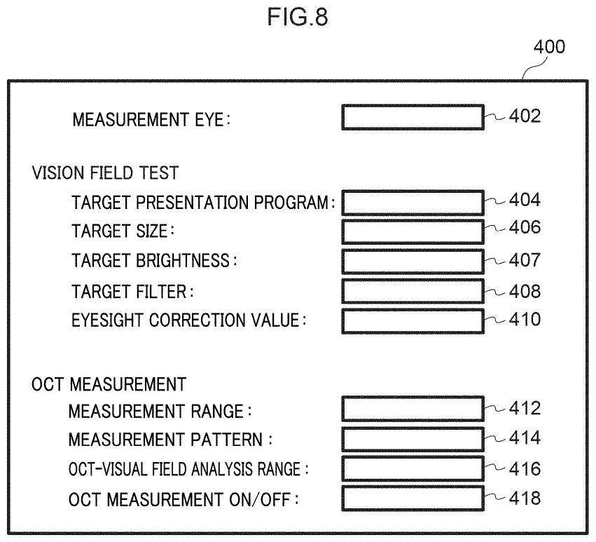

At step 341 of FIG. 7, the processing section 208 displays a measurement condition input screen 400 illustrated in FIG. 8 on the display 32.

As illustrated in FIG. 8, the measurement condition input screen 400 is provided with a measurement eye input section 402, and, for input of various items needed in order to perform vision field test, a target presentation program stipulation input section 404, a target size input section 406, a target brightness stipulation input section 407, a target filter stipulation input section 408, and an eyesight correction value input section 410. Moreover, the measurement condition input screen 400 is also provided with, for input of various items needed in order to perform OCT measurement, a measurement range input section 412, a measurement pattern input section 414, an OCT-visual field analysis range input section 416, and an OCT measurement ON/OFF setting input section 418. Note that each of the input sections (from section 402 to section 418) displays a pull-down menu, and an operator inputs the stipulated data by selecting the desired content from the displayed pull-down menu.

At step 342, the processing section 208 sets the measurement eye according to the input by the operator. More specifically, the operator inputs data stipulating either the left eye or the right eye to the measurement eye input section 402, and at step 342, the processing section 208 receives the data for the left eye or the right eye as input to the measurement eye input section 402 in order to set the measurement eye.

At step 344, the processing section 208 determines whether or not OCT measurement is to be performed based on the content input to the OCT measurement ON/OFF setting input section 418. Affirmative determination is made at step 344 in cases in which OCT measurement has been set by the operator, and so at step 346 the processing section 208 sets a flag F to 1, and at step 350 the processing section 208 sets the OCT measurement conditions before proceeding to step 352.

Specifically, at step 350 the processing section 208 sets an OCT measurement range as input to the measurement range input section 412. For example, the processing section 208 receives data stipulating “all”, or stipulating a range centered on the position of the optical axis of the fundus, for example a 9 mm×9 mm range or a 12 mm×12 mm range, in order to set the OCT measurement range. Moreover, the processing section 208 receives a measurement pattern for OCT measurement as input to the measurement pattern input section 414, and sets this as the measurement pattern. The measurement pattern is a measurement light scan pattern, and examples thereof include linear scanning, radial scanning, and circular scanning. Furthermore, the processing section 208 also receives the OCT-visual field analysis range as input to the OCT-visual field analysis range input section 416, and sets this as the OCT-visual field analysis range.

The OCT-visual field analysis range is a range in the OCT measurement range over which combined analysis including vision field test is to be performed. Explanation follows regarding an example of a case in which the OCT measurement range and the range of vision field test match each other.

In cases in which OCT measurement has not been set by the operator at step 344, the processing section 208 sets the flag F to 0 at step 348, and then the setting processing proceeds to step 352.

At step 352, the processing section 208 receives the eyesight correction value input to the eyesight correction value input section 410 and sets this as the eyesight correction value. Note that lens strength may be set instead of an eyesight correction value.

At step 354, the processing section 208 receives the stipulated target presentation program as input to the target presentation program stipulation input section 404, and sets the stipulated target presentation program.

Explanation follows regarding the target presentation program. The target presentation device 44 presents the targets by projecting the targets at plural different positions on the hemispherical inner face of the target presentation section 52 at different timings. The target presentation program is a program to stipulate the presentation position of each target from out of the plural targets, a presentation light intensity of each of the plural targets, and a presentation duration for presentation of each of the targets.

The target presentation positions are stipulated as target presentation positions in respective regions as measured from the center of the hemispherical inner face of the target presentation section 52 (inner face center) to plural different angles about an optical axis position, namely a line connecting the center of the hemisphere of the target presentation section 52 (sphere center) and the inner face center in a flat plane (horizontal plane (Z-X flat plane) passing through both the inner face center and the sphere center. The plural different angles are, for example, a first angle such as 20°, a second angle such as 40°, and a third angle such as 60°. Polar coordinates (r, θ, φ) indicating coordinates on the hemispherical inner face or scanner angle information may also be employed for the target presentation positions.

The target presentation light intensity is a brightness with which a target is presented on the hemispherical inner face of the target presentation section 52.

The presentation duration is a continuation duration of target presentation.

The target presentation pattern is set by the stipulated target presentation program.

At step 356, the processing section 208 receives the target size as input to the target size input section 406, and sets this as the target size.

At step 358, the processing section 208 receives the target brightness as input to the target brightness stipulation input section 407 and sets this as the target brightness. Note that setting of the target brightness is not limited to inputting the target brightness to the target brightness stipulation input section 407 in this manner, and may be set automatically in the following manner. For example, in cases in which an OCT image has already been acquired and the retinal thickness is already known for the examinee, the processing section 208 may use a prescribed calculation formula to calculate a sensitivity for light perception based on the previously calculated retinal thickness, and the age, gender, and race of the examinee. The processing section 208 then automatically sets the target brightness based on the calculated sensitivity.

At step 360, the processing section 208 receives the target filter as input to the target filter stipulation input section 408, and sets this as the target filter.

Note that the sequence of the processing of the respective steps from step 352 to step 360 is not limited to the above sequence.

Setting of the measurement conditions is completed by performing the processing from step 342 to step 360, after which the vision field test and OCT image acquisition processing proceeds to step 306 of FIG. 6.

At step 306 of FIG. 6 the processing section 208 determines whether or not the examination eye 10 has been detected by the external camera 46. In cases in which the examination eye 10 has not been detected by the external camera 46, step 306 is repeated until the examination eye 10 has been detected. Once the operator has input the measurement conditions and these measurement conditions have been set, the operator then asks the examinee to rest their chin on a non-illustrated prescribed chinrest of the ophthalmic device 110A in order to test the examination eye 10. When the examination eye 10 is to be tested the examination eye 10 is detected by the external camera 46. In cases in which determination is made that the examination eye 10 has been detected by the external camera 46, at step 308 the processing section 208 determines whether or not the flag F has been set to 1.

In cases in which the flag F has been determined to be set to 1 at step 308, at step 310 the processing section 208 presents the fixation target, as illustrated in FIG. 9, by illuminating the fixation target presentation section 42.

At step 312, the processing section 208 controls the external camera 46 so as to acquire an alignment image (anterior eye portion image) to determine whether or not an OCT image of the measurement range can be acquired.

Note that the alignment image is displayed on the display 32, and the operator checks the displayed alignment image, and adjusts the chinrest such that the position of the examination eye 10 is positioned at a position enabling an OCT image of the measurement range to be acquired. Note that such adjustment may be adjustment performed by the operator speaking to the examinee, moving the chinrest, or the like.

At step 314, in order to generate an OCT image, the second lens driving control section 206 drives the second lens driving device 40 so as to adjust the position of the second lens 14. The first lens driving control section 204 then adjusts the position of the first lens 12 according to the adjusted position of the second lens 14. Focus adjustment is performed by this operation.

At step 316, the processing section 208 controls the scanner 48G of the OCT imaging device 48 to acquire an OCT image. The acquisition range of the OCT image corresponds to the OCT measurement range set at step 350 of FIG. 7. Note that when the OCT image has been acquired at step 316, illumination of the fixation target presentation section 42 is ended.

In cases in which the flag F is determined not to be set to 1 at step 308, the processing of step 310 to step 316 is skipped. The OCT measurement is thus skipped and the vision field test is started.

At step 318, the processing section 208 presents the fixation target by illuminating the fixation target presentation section 42 as described above.

At step 320, in order to perform the vision field test and OCT image acquisition, the second lens driving control section 206 drives the second lens driving device 40 so as to adjust the position of the second lens 14. Next, the first lens driving control section 204 drives the first lens driving device 38 so as to adjust the position of the first lens 12.

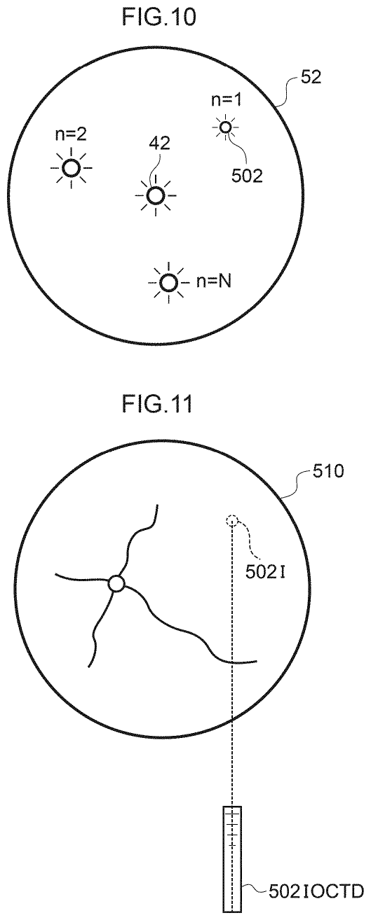

At step 322, the processing section 208 sets a variable n of the target presentation program to 0. The variable n is a variable used to identify presentation positions of the targets. At step 324 the processing section 208 increments the variable n by 1. At step 326, the processing section 208 controls the target presentation device 44 so as to present the target at a position n on the hemispherical inner face of the target presentation section 52 as identified by the variable n. Thereby, for example, the target 502 is presented at the position n on the hemispherical inner face of the target presentation section 52, as illustrated in FIG. 10. A total N is a maximum value of variable n, and is the number of targets needed to perform vision field testing (the number of targets to be projected).

When the target 502 is presented, light from the target 502 passes from the target presentation section 52, along the optical path 44P, through the first lens 12, the optical path combining section 16, and the second lens 14, before reaching the retina of the examination eye 10. The examinee switches the perception switch 30 ON if the examinee perceives the light reaching the retina of the examination eye 10. However, the examinee does not switch the perception switch 30 ON if the examinee does not perceive the light reaching the retina of the examination eye 10.

At step 328, the processing section 208 checks whether or not a perception signal has been sent from the perception switch 30. More specifically, the perception signal from the perception switch 30 is input to the control device 20 in cases in which the target 502 is presented at the position n (for example n=1) on the hemispherical inner face of the target presentation section 52 as illustrated in FIG. 10, and the perception switch 30 has been switched ON. In cases in which the perception signal has been input, determination may be made that light from the target 502 has been perceived at a position 5021 (see also FIG. 11) of the retina 510 corresponding to the position n where the target 502 is presented. In such cases determination may be made that there is no problem present at the position 5021 of the retina 510, and the processing section 208 accordingly does not store data for the position 5021.

Note that the position 5021 of the retina 510 corresponding to the position n where the target 502 is presented is a position for which a positional relationship between the position of the optical axis of the retina of the examination eye 10 and the position 5021 corresponds to a positional relationship between the center of the hemispherical inner face (inner face center) of the target presentation section 52 and the position n where the target 502 is presented.

More specifically, the first lens 12 forms an intermediate image of the target 502 between the first lens 12 and the second lens 14. This intermediate image is formed by the second lens 14 into an image at the position 5021 of the retina 510 corresponding to the position n where the target 502 is presented.

However, the perception signal is not input in cases in which light reaches the retina of the examination eye 10 but the perception switch 30 is not switched ON. Cases in which the perception signal is not input indicate that the light from the target 502 has not been perceived at the position 5021 of the retina 510. In such cases, determination may be made that there is a problem at the position 5021 of the retina 510, and the processing section 208 accordingly stores data for the position 5021. Note that regarding the perception signal, for each target, zero (0) may be stored in cases in which the perception switch 30 is not switched ON, and one (1) may be stored in cases in which the perception switch 30 is switched ON.

At step 330, the processing section 208 controls the target presentation device 44 so as to end presentation of the target.

At step 332, the processing section 208 controls the OCT imaging device 48 so as to acquire an OCT image of the position 5021 corresponding to the position n of the retina 510. OCT image data 502 IOCTD of the retina with reference to the position 5021 of the retina 510 is acquired by the processing of step 332, as illustrated in FIG. 11.

Note that a configuration may be adopted in which at step 332, the processing section 208 controls the OCT imaging device 48 so as to acquire data of an OCT image of the retina either for a one-dimensional region including the position 5021 (for example, a 1 mm line segment passing through the target) or for a two-dimensional region including the position 5021 (for example, a 1 mm×1 mm range centered on the target). Moreover, configuration may be made such that step 332 is executed only in cases in which the perception signal has not been switched ON.

At step 334, the processing section 208 determines whether or not the variable n is equal to the total N of the stipulated targets for presentation as defined by the stipulated target presentation program. Processing returns to step 324 when the variable n is not determined to be equal to the total N since this means that there are still targets stipulated for presentation by the target presentation program that have not yet been presented. The above processing (step 324 to step 334) is then repeated.

In cases in which determination at step 334 is that the variable n is equal to the total N, this means that the number of targets as defined by the target presentation program have already been presented, and so at step 336 the processing section 208 creates a visual field defect map based on the total N target positions and the perception signals corresponding to the respective targets.

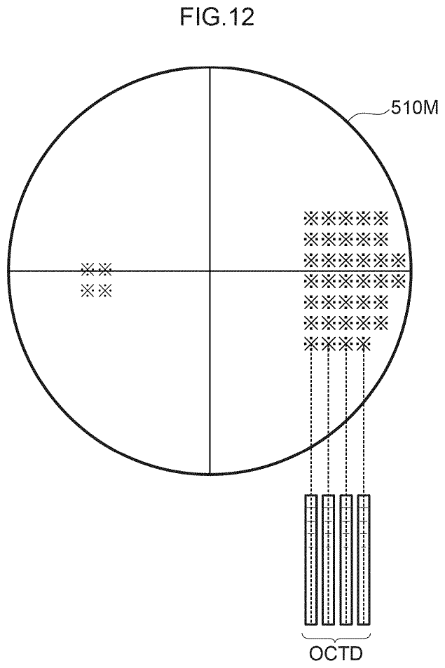

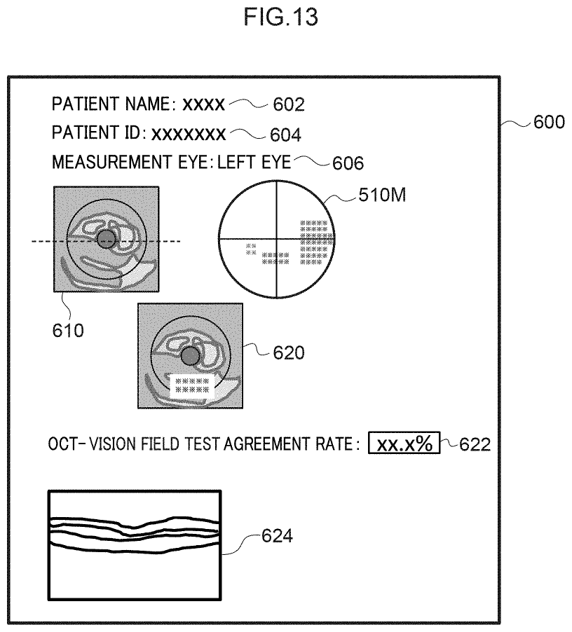

FIG. 12 illustrates a visual field defect map 510M. The visual field defect map 510M is a vision field test result image display field displayed on a report display screen 600, illustrated in FIG. 13. As described above, a position of the retina 510 corresponding to a target position is stored in cases in which the perception switch 30 was not switched ON in response to presenting the target (step 328). The visual field defect map 510M is a map, as illustrated in FIG. 12, in which marks (such as “X”) are displayed for stored positions where the perception signal was absent, indicating that there is a problem with the optic nerve at that position. Note that in the example illustrated in FIG. 12 there are comparatively many visual field defect regions and therefore comparatively many marks displayed on the right side of the optical axis, while comparatively few marks are displayed on the left side of the optical axis.

An OCT image is acquired for each position for positions corresponding to the target presentation positions (see step 332).

The processing section 208 performs OCT image analysis at step 338, and at step 340 either displays measurement data or creates a report. In the OCT image analysis, the obtained OCT volume data is segmented into layers configuring the retina, and processing is performed thereon to quantify the thickness of the stratum opticum.

Explanation follows regarding the measurement data and report created and displayed based on the OCT image analysis results at step 338, with reference to FIG. 13.

As illustrated in FIG. 13, the report display screen 600 includes an examinee name display field 602 to display the name of the examinee, an examinee ID display field 604 to display the examinee ID, and an examination eye display field 606 to display the measurement eye. Based on the examinee information input at step 302, the processing section 208 displays the examinee name in the examinee name display field 602, displays the examinee ID in the examinee ID display field 604, and displays text regarding the measurement eye (for example “left eye”) in the examination eye display field 606.

A color-coded retinal thickness image display field 610 is also provided on the report display screen 600, and a color-coded retinal thickness image is displayed in the color-coded retinal thickness image display field 610. The color-coded retinal thickness image displayed in the color-coded retinal thickness image display field 610 is an image in which retinal thickness, which has been calculated by the processing section 208 based on the OCT image data of the OCT images acquired for each position at step 332, is converted into a color-coded image of the retina based on the calculated thickness of the retina. More specifically, a first color (for example yellow) is employed to display positions where the thickness of the retina is thinner than a prescribed thickness, and a second color (for example blue) is employed to display positions where the thickness of the retina is the prescribed thickness or greater. The prescribed thickness corresponds to retinal thickness each position in a healthy person, and is defined according to age, gender, and race.

A vision field test result image display field is also provided on the report display screen 600. The visual field defect map 510M is displayed in the vision field test result image display field.

As described above, an OCT image is acquired for each position for positions corresponding to the target presentation positions (step 332). A configuration may be adopted in which clicking on a mark indicating the presence of a problem with the optic nerve (“X” in the example described above) displays a numerical value corresponding to the thickness of the stratum opticum, as obtained from the OCT image data.

An OCT image-vision field test result combined display field 620 is provided on the report display screen 600. An image combining the color-coded retinal thickness image display field 610 and the visual field defect map 510M is displayed in the OCT image-vision field test result combined display field 620.

An OCT-vision field test agreement rate display field 622 is provided on the report display screen 600. The OCT-vision field test agreement rate is, for example, a ratio of the number of individual positions where the thickness of the retina is thinner than the prescribed thickness and there is a defect present in the visual field, with respect to the number of individual positions where the thickness of the retina is thinner than the prescribed thickness. The processing section 208 calculates the OCT-vision field test agreement rate and displays the OCT-vision field test agreement rate in the OCT-vision field test agreement rate display field 622.

An OCT image display field 624 is provided on the report display screen 600, and an OCT image of regions of the retina where visual field defects are present is displayed on the OCT image display field 624 in the report display screen 600 based on the OCT image data acquired at step 332.

In the example described above, the ophthalmic device 110A is configured such that the report display screen 600 is displayed on the display 32, however technology disclosed herein is not limited thereto. For example, a configuration may be adopted in which the processing section 208 transmits data obtained by the processing of steps 302, 304, 316, and step 322 to step 334 to the server 140, and the server 140 executes the processing from step 336 to step 340. In such cases configuration may be made such that the processing of step 340 is performed by the viewer 150. Transmission instruction data, including an examinee ID, is transmitted to instruct transmission of the data of the report display screen 600 from the server 140 to the viewer 150. On receipt of the transmission instruction data, the server 140 transmits the data of the report display screen 600 corresponding to the examinee ID to the viewer 150. The viewer 150 then displays the report display screen 600 as illustrated in FIG. 13.

In the present exemplary embodiment as described above, since the ophthalmic device 110A includes both the vision field testing section 110AA and the measuring section 110AB, both vision field testing and OCT imaging can be performed using a single device. This accordingly enables the burden on the examinee to be lessened since the examinee does not need to move from a device for vision field testing to a device for OCT imaging.

Moreover, in the present exemplary embodiment, the optical path combining section 16 combines the optical path 44P of the vision field testing optical system and the optical path 48P of the measurement optical system of the OCT imaging device 48. This enables a device that performs both vision field testing and OCT imaging to be made more compact. The ophthalmic device 110A can accordingly be made smaller.

Furthermore, the present exemplary embodiment enables both the presence or absence of a visual field defect and the retinal thickness based on the OCT image (the OCT volume data) to be ascertained at any given position for each of the retinal positions corresponding to the plural different target presentation positions. Normally in the case of glaucoma, thinning of the optic nerve cells occurs where visual field defects are present in the retina. Thus the ability to ascertain an association between retinal optic nerve cells exhibiting visual field defects and the thickness thereof enables a more quantitative diagnosis of glaucoma to be made. Moreover, in cases in which a visual field defect is present but the thickness of the optic nerve cells of the retina where the visual field defect is present is not thinner than the prescribed thickness, the visual field defect can be diagnosed as having arisen from a pathological lesion other than glaucoma, enabling a new pathological lesion to be presented.

Moreover, the present exemplary embodiment enables quick completion of the vision field testing and the OCT image acquisition.

### Second Exemplary Embodiment

Next, explanation follows regarding a second exemplary embodiment. Since the second exemplary embodiment has substantially the same configuration as the first exemplary embodiment, explanation follows regarding elements that differ therefrom.

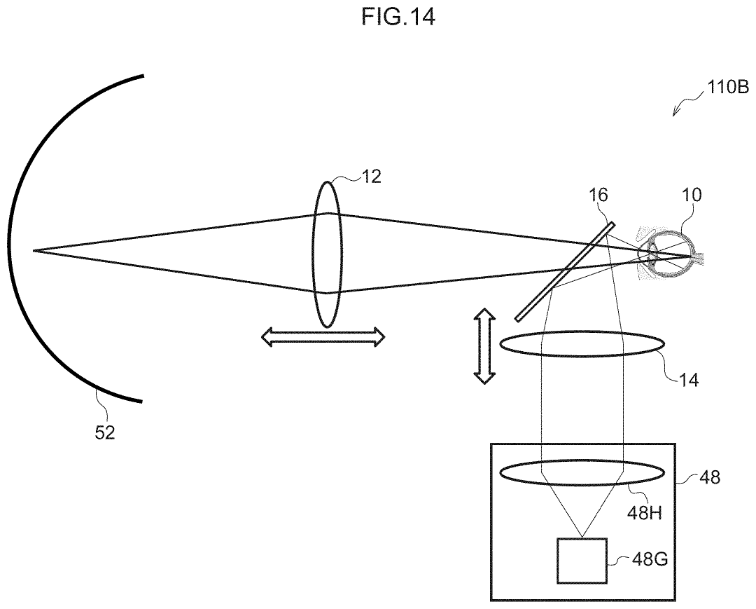

FIG. 14 illustrates a configuration of an ophthalmic device 110B of the second exemplary embodiment. As illustrated in FIG. 14, the ophthalmic device 110B differs in the point that the second lens 14 is provided between the optical path combining section 16 and the OCT imaging device 48.

Providing the second lens 14 between the optical path combining section 16 and the OCT imaging device 48 in this manner enables a claustrophobic sensation of the examinee caused by the second lens 14 to be alleviated.

### Third Exemplary Embodiment

Next, explanation follows regarding a third exemplary embodiment. Since the third exemplary embodiment has substantially the same configuration as the second exemplary embodiment, explanation follows regarding only elements differing therefrom.

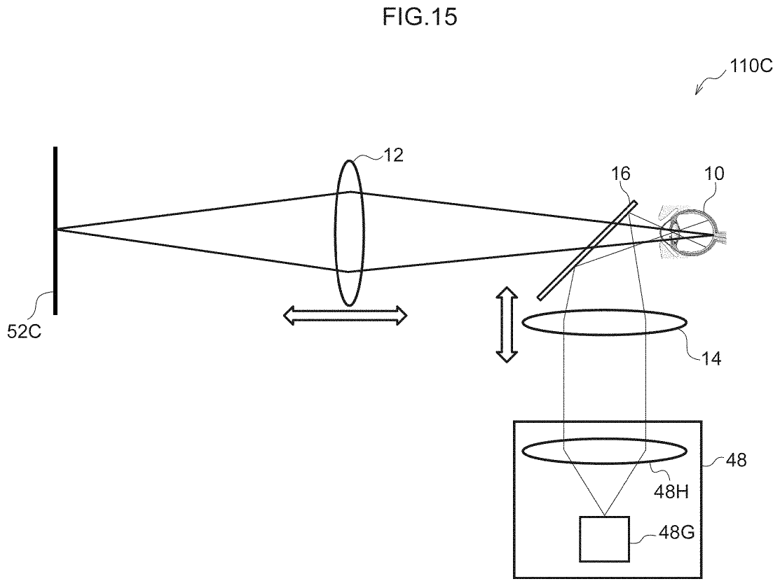

A configuration of an ophthalmic device 110C of the third exemplary embodiment is illustrated in FIG. 15. As illustrated in FIG. 15, the ophthalmic device 110C of the third exemplary embodiment includes a target presentation section 52C. The target presentation section 52C includes the functionality of the fixation target presentation section 42 and the functionality of the target presentation device 44 of the first exemplary embodiment. Namely, the target presentation section 52C includes non-illustrated light emitting devices laid out in a matrix. The target presentation section 52C may be configured by a liquid crystal display device or an organic EL display device. Moreover, a fixation target display lamp is provided on the optical axis for displaying the fixation target. The target presentation section 52C is controlled such that light emitting devices emit light at positions defined by a visual field presentation program.

### Fourth Exemplary Embodiment

Next, explanation follows regarding a fourth exemplary embodiment. Since the fourth exemplary embodiment has substantially the same configuration as the third exemplary embodiment, explanation follows regarding only elements differing therefrom.

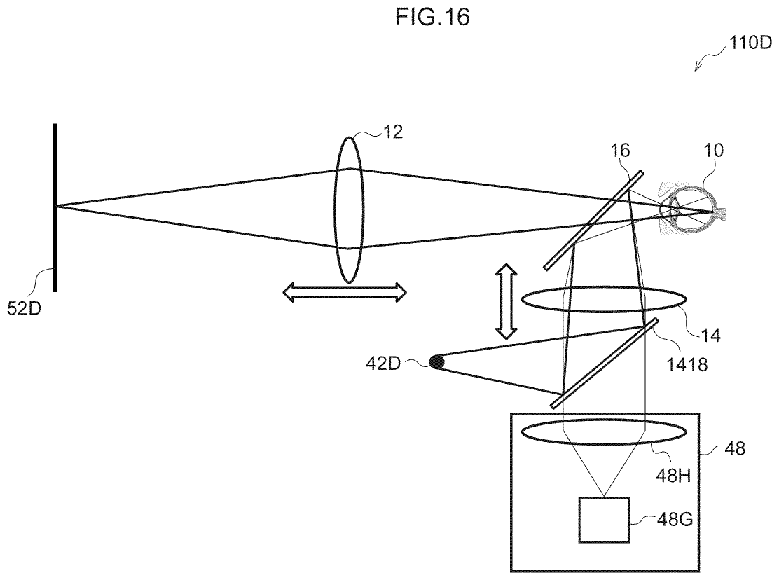

FIG. 16 illustrates a configuration of an ophthalmic device 110D of the fourth exemplary embodiment. A target presentation section 52D of the ophthalmic device 110D includes the functionality of the target presentation device 44, but does not include the functionality of the fixation target presentation section 42 of the first exemplary embodiment. A point light source fixation target presentation section 42D is provided in the ophthalmic device 110D of the fourth exemplary embodiment. Abeam splitter 1418 is provided between the second lens 14 and the OCT imaging device 48. Light from the point light source fixation target presentation section 42D is reflected by the beam splitter 1418 on the second lens 14 side, and reaches the retina of the examination eye 10 via the second lens 14 and the optical path combining section 16. The examinee perceives the fixation target as being positioned on the optical axis of the target presentation section 52D.

The fixation target presentation section 42D is not limited to being a point light source, and may be a display performed by a display unit.

### MODIFIED EXAMPLES

Next, explanation follows regarding various modified examples of technology disclosed herein.

### First Modified Example

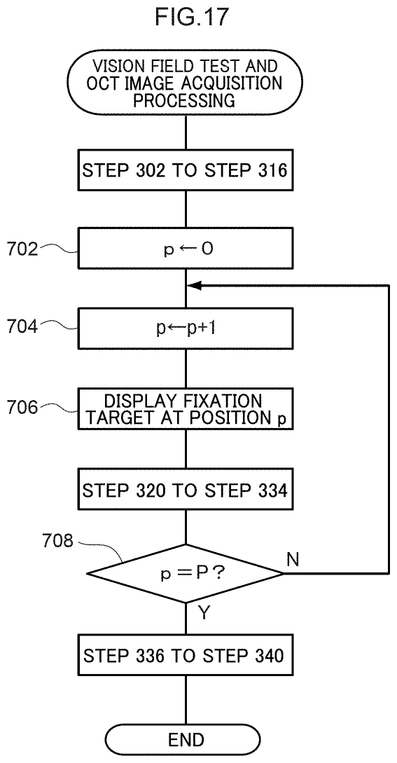

FIG. 17 illustrates a flowchart of vision field test and OCT image acquisition processing of a first modified example. As illustrated in FIG. 17, after executing step 302 to step 316 of the vision field test and OCT image acquisition processing of the first modified example, the processing section 208 sets a variable p employed to identify fixation target positions to 0 at step 702, and increments the variable p by one at step 704. At step 706 the processing section 208 presents the fixation target at a position p as identified from the variable p, and then executes step 320 to step 334.

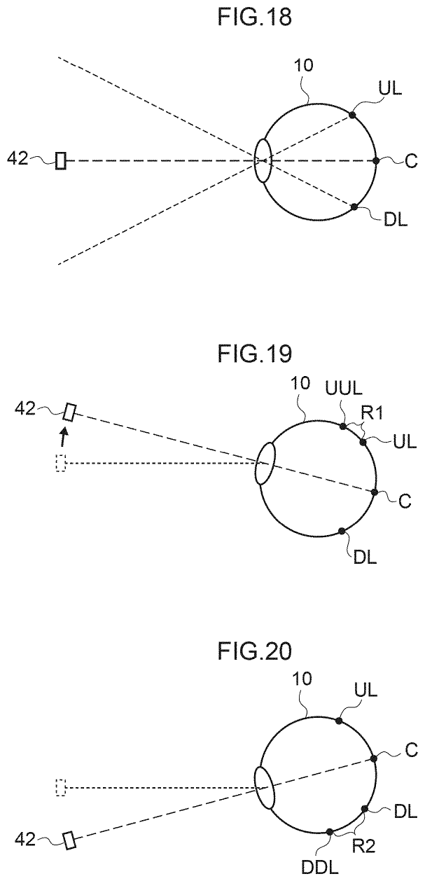

When the variable p=1, this identifies a position at the optical axis. Thus the fixation target is presented on the optical axis when the variable p=1. In step 320 to step 334 when the variable p=1, as illustrated in FIG. 18, visual field defects on the retina are measured for a region spanning from an upward limit position UL of the retina to a downward limit position DL of the retina, and OCT images are also acquired for this region. Visual field defect measurements are not performed, OCT images are not acquired, for the retina at positions lying above the position UL or below the position DL.

At step 708, the processing section 208 determines whether or not the variable p is equal to a total number P of fixation target positions. The present processing returns to step 704 when determination is not made that the variable p is equal to the total P.

When the variable p=2, this identifies a position at a prescribed distance above the optical axis, as illustrated in FIG. 19. When the position of the fixation target presentation section 42 is moved to the position the prescribed distance above the optical axis, the optical axis of the examination eye 10 is shifted upward. This enables measurement of visual field defects and acquisition of OCT images in a region R1 spanning as far as a position UUL above the position UL.

Moreover, when the variable p=3, this identifies a position below the optical axis, as illustrated in FIG. 20. When the variable p=3, at step 320 to step 334 the fixation target presentation section 42 is moved to a position the prescribed distance below the optical axis, and the optical axis of the examination eye 10 is shifted downward. This enables measurement of visual field defects and acquisition of OCT images in a region R2 spanning as far as a position DDL below the position DL.

Note that when the variable p=4, this identifies a position a prescribed distance to the right of the optical axis from the perspective of the examinee, and when the variable p=5, this identifies a position a prescribed distance to the left of the optical axis from the perspective of the examinee.

In cases in which determination is made at step 708 that the variable p is equal to the total P (this being 5 in the above example), this indicates that step 336 to step 340 have been executed for each of the positions identified by the variable p.

The first modified example configured in this manner enables vision field testing and OCT image acquisition to be performed over a wider range of the retina.

Explanation follows regarding a method to present the fixation target at the positions identified by the variable p, taking the first exemplary embodiment as an example.

A hole is provided in the hemispherical inner face of the target presentation section 52 at each of the positions identified by the variable p, and fixation target presentation sections 42 are provided at positions corresponding to each of the provided holes. At step 706 the fixation target presentation section 42 at the position identified by the variable p is illuminated. Alternatively, a configuration may be adopted in which a single fixation target presentation section 42 is provided, and the fixation target presentation section 42 is then moved to the respective positions identified by the variable p and illuminated thereat.

Furthermore, a configuration may be adopted in which step 310 to step 316 of FIG. 6 are executed for presentation of the fixation target at each of the positions identified by the variable p. Moreover, a configuration may be adopted in which a thickness is acquired for each retinal position from the results of executing step 310 to step 316 for the presentation of the fixation target at each of the positions identified by the variable p, and the vision field testing and OCT image acquisition (step 318 to step 334) are executed for locations where this thickness is a prescribed thickness or thinner.

### Second Modified Example

Explanation follows regarding a second modified example. In the second modified example, the measuring section 110AB includes the functionality of the vision field testing section 110AA. More specifically, the OCT imaging device 48 includes a visible light source that emits visible light as a target, and includes a non-illustrated beam splitter that guides visible light from the visible light source along an optical path 48P. The vision field testing section 110AA is omitted in the second modified example. The optical path 44P and the optical path 48P of the visible light from the visible light source split by the beam splitter are combined in the OCT imaging device 48. The first lens 12 and the first lens driving device 38 are accordingly also omitted, and the focus adjustment and diopter adjustment during OCT image acquisition are performed using the second lens 14 alone.

The fixation target is guided from an additional separate light source to the examination eye 10 along a separate optical path to the optical path 48P.

The second modified example enables a device that performs both vision field testing and OCT imaging to be made more compact, and enables a smaller ophthalmic device to be achieved.

Furthermore, the processing to present the target (step 326) and the processing to acquire an OCT image (step 332) can both be executed at the same time in the second modified example. This enables quicker completion of the vision field testing and OCT image acquisition.

### Third Modified Example

Explanation follows regarding a third modified example.

In the first exemplary embodiment, the target presentation (step 326), the processing to check whether or not the perception switch has been switched ON (step 328), the processing to end the target presentation (step 330), and the processing to acquire an OCT image (step 332) are executed in this sequence. Thus the OCT image is acquired in a state in which visible light is not being irradiated onto the retina. This enables an OCT image to be obtained in a state in which the retina is not responding to visible light.

In contrast thereto, step 330 of FIG. 6 is executed after step 332 in the third modified example. The target presentation (step 326), the processing to check whether or not the perception switch has been switched ON (step 328), the processing to acquire an OCT image (step 332), and the processing to end the target presentation (step 330) are executed in this sequence. The OCT image is accordingly acquired in a state in which visible light is being irradiated onto the retina. This accordingly enables an OCT image to be obtained in a state in which the retina is responding to visible light.

Note that a configuration may be adopted that includes a first mode in which step 332 of FIG. 6 is executed after step 330, and a second mode in which step 330 is executed after step 332, as in the third modified example. In such cases both the first mode and the second mode may be executed, or one mode selected from out of the first mode and the second mode may be executed.

In cases in which both the first mode and the second mode are executed, the following processing may be implemented. First, a first thickness of each retinal position may be acquired from an OCT image acquired in a state in which the retina is not responding to visible light, and then a second thickness of each retinal position may be acquired from an OCT image acquired in a state in which the retina is responding to visible light. A difference between the first thickness and the second thickness may then be calculated for each position.

### Fourth Modified Example

The following additional processing may be executed as a fourth modified example in the first exemplary embodiment through to the third modified example.

Differences between past vision field testing results and the latest results for a current occasion are acquired in order to find a visual field defect progression status. Moreover, differences between past retinal thickness results and the latest results for the current occasion are acquired based on OCT images to find changes in retinal thickness.

### Fifth Modified Example

Instead of the OCT imaging device 48, a combination may be made with another eye measurement/testing device, such as a fundus camera or an anterior eye portion measurement device. The OCT imaging device 48 may be configured to perform eyeball tracking using a position of the examination eye 10 detected by the external camera 46.

### Sixth Modified Example

Although in the above exemplary embodiments examples have been described of an ophthalmic system 100 including an ophthalmic device 110A (through to ophthalmic device 110D), a server 140, and a viewer 150, the technology disclosed herein is not limited thereto. For example, the ophthalmic device 110A (through to ophthalmic device 110D) may also include the functionality of at least one of the server 140 or the viewer 150. For example, the server 140 may be omitted in cases in which the ophthalmic device 110A (through to ophthalmic device 101D) includes the functionality of the server 140. Moreover, the viewer 150 may be omitted in cases in which the ophthalmic device 110A (through to ophthalmic device 110D) includes the functionality of the viewer 150. Furthermore, a configuration may be adopted in which the server 140 is omitted, and the viewer 150 executes the functionality of the server 140.

### OTHER MODIFIED EXAMPLES

The processing described in the first exemplary embodiment to the sixth modified example is merely an example thereof. Obviously, unnecessary steps may be omitted, new steps may be added, and the sequence of processing may be changed within a range not departing from the spirit thereof.

Moreover, although in the first exemplary embodiment to the sixth modified example described above examples have been given of cases in which data processing is implemented by a software configuration utilizing a computer, the technology disclosed herein is not limited thereto. For example, instead of a software configuration utilizing a computer, the data processing may be executed solely by a hardware configuration of field programmable gate arrays (FPGA) or application specific integrated circuits (ASIC). Alternatively, a portion of processing in the data processing may be executed by a software configuration, and the remaining processing may be executed by a hardware configuration.

What is claimed is:

1. An ophthalmic device comprising:
a vision field testing section including a target presentation section and a vision field test optical system and configured to perform a vision field test on an examination eye;
a measuring section including a measurement optical system to perform optical coherence tomography measurements on the examination eye, and a measurement unit configured to generate a tomographic image of the examination eye;
an optical path combining section provided between the examination eye and the target presentation section and configured to combine a first optical path of the vision field test optical system and a second optical path of the measurement optical system;
a first lens provided between the optical path combining section and the target presentation section and configured to perform diopter adjustment;
a second lens provided between the optical path combining section and either a position of the examination eye or the measurement unit and employed in focus adjustment;
a first driving section configured to move the first lens;
a second driving section configured to move the second lens; and
a control section configured to control the first driving section and the second driving section.

2. The ophthalmic device of claim 1, wherein when the vision field test is being performed, the control section drives the first driving section and the second driving section to adjust positions of the first lens and the second lens.

3. The ophthalmic device of claim 1, wherein when the vision field test is being performed, the control section moves at least the first driving section based on an eyesight correction value for the examination eye.

4. The ophthalmic device of claim 1, wherein when the tomographic image is being generated, the control section drives the second driving section so as to adjust a position of the second lens.

5. The ophthalmic device of claim 1, wherein the first lens is positioned on the first optical path.

6. The ophthalmic device of claim 1, further comprising a fixation target presentation section commonly employed both when the vision field test is being performed and when the tomographic image is being generated.

7. The ophthalmic device of claim 1, wherein the target presentation section is a dome onto which targets are projected by the vision field test optical system.
